(19) United States
(12) Patent Application Publication
(10) Pub. No.: US 2024/0407389 A1
(43) Pub. Date: Dec. 12, 2024

(54) METHODS FOR NUTRITIONAL ENHANCEMENT OF FUNGAL MYCELIUM

(71) Applicant: Emergy Inc., Boulder, CO (US)

(72) Inventors: Tracy B. YATES, Arvada, CO (US); Justin WHITELEY, Lafayette, CO (US)

(21) Appl. No.: 18/694,787

(22) PCT Filed: Sep. 23, 2022
(86) PCT No.: PCT/US2022/044595

§ 371 Date: Mar. 22, 2024

Related U.S. Application Data
(60) Provisional application No. 63/247,652, filed on Sep. 23, 2021.

(51) Int. Cl. A23J 3/22; A23J 1/00; A23L 31/00; C12N 1/14; C12R 1/645

(52) U.S. Cl. CPC A23J 3/227; A23J 1/008; A23L 31/00; C12N 1/14; C12R 2001/645

(57) ABSTRACT

A method of forming an edible meat substitute product can include providing a growth media having an iron amount in a range of 0.1 mg/L to 10 mg/L and growing fungal cells in the growth media such that the fungal cells include a mycelium mass. The mycelium mass may have an iron content greater than or equal to 5 mg of iron per 100 g of mycelium mass. The method can include separating the mycelium mass from the growth media and exposing the mycelium mass to a light having a wavelength in a range of 100 nanometers to 400 nanometers for a period of time so as to form an enhanced mycelium product having a vitamin D content greater than 1 μg per 100 grams of the enhanced mycelium product.

### TECHNICAL FIELD

This application claims the benefit of U.S. Provisional Application No. 63/247,652, filed on Sep. 23, 2021, the contents of which are incorporated herein by reference in their entirety.

The present disclosure relates generally to the field of fungal mycelium based edible meat substitute products.

### BACKGROUND

Demand for edible products that can provide a high protein content which is drawn from a non-animal source is increasing. Driven by increasing awareness of personal health, edible products that include non-animal sourced components such as proteins and fibers are considered as a healthier alternative to animal protein-based products. In particular, there is growing demand for edible meat substitutes that mimic meat in its composition and texture but are composed of non-animal components, which can reduce reliance on animals such as cows, chicken, and pigs, and reduce the carbon footprint posed by such animals. Furthermore, fortification of food products can be used to address potential deficiencies in essential nutrients such as vitamin D and iron. It is estimated that at least one billion people worldwide suffer from vitamin D deficiency. Similarly, there is a high incidence of iron deficiency in women and infant children.

### SUMMARY

Embodiments described herein relate generally to the methods of forming nutritionally enhanced mycelium products for obtaining edible meat substitute products that resemble animal meat in their texture and morphology, and particularly, to methods of forming enhanced mycelium edible products that include Vitamin D and/or iron.

In some embodiments, a method of forming an edible meat substitute product includes providing a mycelium product comprising fungal mycelium and exposing the mycelium product to a light having a wavelength in a range of 100 nanometers to 400 nanometers for a first period of time so as to form an enhanced mycelium product having a vitamin D content of greater than 1 μg per 100 grams of the enhanced mycelium product.

In some embodiments, a method of forming an edible meat substitute product includes providing a first growth media having a first iron amount in a range of 0.1 mg/L to 10 mg/L. The method further includes growing fungal cells in the first growth media such that the fungal cells produce a mycelium mass. The mycelium mass has an iron content greater than or equal to 5 mg of iron per 100 g of mycelium mass. In some embodiments, the mycelium mass has an iron content of from about 5 mg of iron per 100 g of mycelium mass to about 15 mg of iron per 100 g of mycelium mass. The method further includes separating the mycelium mass from the first growth media.

In some embodiments, a method of forming an edible meat substitute product includes providing a growth media having a first iron amount in a range of 0.1 mg/L to 10 mg/L. The method further includes growing fungal cells in the growth media such that the fungal cells produce a mycelium mass. The mycelium mass has an iron content greater than or equal to 5 mg of iron per 100 g of mycelium mass. In some embodiments, the mycelium mass has an iron content of from about 5 mg of iron per 100 g of mycelium mass to about 15 mg of iron per 100 g of mycelium mass. The method further includes separating the mycelium mass from the growth media. The method further includes exposing the mycelium mass to a light having a wavelength in a range of 100 nanometers to 400 nanometers for a period of time so as to form an enhanced mycelium product having a vitamin D content of greater than 1 μg per 100 grams of the enhanced mycelium product.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several implementations in accordance with the disclosure and are therefore, not to be considered limiting of its scope, the disclosure will be described with additional specificity and detail through use of the accompanying drawings.

FIG. 1 is a flow chart of a method for increasing the vitamin D content of a mycelium product, in accordance with some embodiments.

FIG. 2 is a flow chart of a method for increasing the iron content of a mycelium mass, in accordance with some embodiments.

FIG. 3 is a flow chart of a method for increasing both the vitamin D content and the iron content of a mycelium product, in accordance with some embodiments.

FIG. 4A is a graph of the vitamin D content of various edible products including a UV treated mycelium product and an untreated mycelium product.

FIG. 4B is a table corresponding to FIG. 4A of the vitamin D content of various edible products including the UV treated mycelium product and the untreated mycelium product.

FIG. 5A is a table of the composition of a control growth media for fungal mycelium, in accordance with some embodiments.

FIG. 5B is a table of the composition of a growth media with 2X iron content for fungal mycelium, in accordance with some embodiments.

FIG. 5C is a table of the composition of a growth media with 5X iron content for fungal mycelium, in accordance with some embodiments.

Reference is made to the accompanying drawings throughout the following detailed description. In the drawings, similar symbols typically identify similar components, unless context dictates otherwise. The illustrative implementations described in the detailed description, drawings, and claims are not meant to be limiting. Other implementations may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### DETAILED DESCRIPTION

Embodiments described herein relate generally to methods of forming nutritionally enhanced mycelium products for obtaining edible meat substitute products that resemble animal meat in their texture and morphology, and particularly, to methods of forming enhanced mycelium edible products that include Vitamin D and/or iron.

Various embodiments described herein provide methods of providing a mycelium product, exposing the mycelium product to a light having a wavelength in a range of 100 nanometers to 400 nanometers, and providing an enhanced mycelium product with an increased vitamin D content. In some embodiments, the wavelength, a distance to the light, and an exposure time to the light can be varied in order to target a desired vitamin D content. In some embodiments, a first side and a second side of the mycelium product can both be exposed to the light to uniformly enhance vitamin D content. In some embodiments, vitamin D content can be increased at multiple points during the production of the enhanced mycelium product.

In at least some embodiments comprising exposing mycelium product to light, the exposure does not cause the production of pigmentation in the mycelium product. In at least some instances, the production of pigmentation in mycelium upon exposing the mycelium to light is undesirable. Accordingly, at least some embodiments comprising exposing mycelium product to light that are described herein constitute an improvement over processes involving exposing mycelium to light that result in the production of pigmentation.

Other embodiments described herein provide methods of providing a growth media with an iron amount for fungal mycelium, growing fungal cells in the growth media to produce a mycelium mass, and separating the mycelium mass with an increased iron content. In some embodiments, the iron amount in the growth media directly corresponds to the iron content of the mycelium mass. In some embodiments, a sugar, a nitrogen-containing compounds, or a phosphate-containing compounds may also be added to the mycelium mass. In some embodiments, multiple growth media may be used to increase iron content or grow the fungal cells for the mycelium mass. Increasing the vitamin D content and/or the iron content of mycelium products provides nutritional enhancement to the mycelium products and results in a healthy meat substitute for consumers.

One parameter in forming edible meat substitute products is providing nutrients in a natural way. Typically, food products are fortified with exogenously derived vitamins and minerals to increase nutritional content. Naturally, mycelium may have no vitamin D and a low iron content (e.g., less than 2 mg/100 grams). Achieving nutritional enhancements with less food processing and through the biological activity of the mycelium mass itself is beneficial for consumers' health and production costs. Thus, there is a need for non-animal protein sources that can facilitate large scale production and adoption of non-animal based edible products as well as provide nutritional benefits.

Various embodiments of the methods for enhancing the nutrition of fungal mycelium may provide one or more benefits including, for example: (1) increasing the nutritional content of the mycelium product using the biological activity of the organism itself rather than by fortifying food formulation; (2) providing a food product with fewer ingredients; (3) reducing the number of chemicals in the mycelium product; (4) providing an avenue for organic certification; (5) addressing vitamin D and iron deficiencies commonly found worldwide; (6) avoiding excess processing of the mycelium product; (7) providing a method that can be applied during multiple points of manufacturing the mycelium product; (8) meeting or exceeding the Recommended Dietary Allowance (RDA) of vitamin D; (9) meeting or exceeding the Recommended Daily Allowance (RDA) of iron; (10) varying light exposure based on desired vitamin D content; (11) varying iron amount based on desired iron content; (12) increasing nutritional content without impacting the growth rate of the mycelium product; and (13) providing the Vitamin D and/or iron intracellularly.

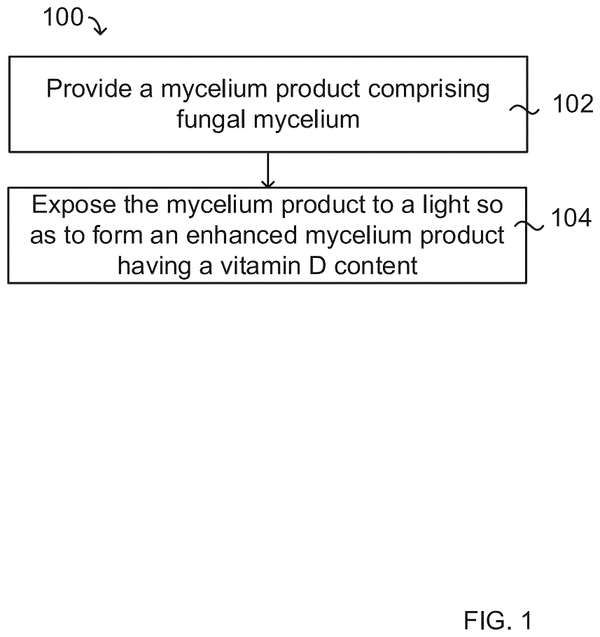

FIG. 1 illustrates a block diagram of an example method 100 for forming an edible meat substitute product, according to an embodiment. The example method 100 may increase the vitamin D content of a mycelium product. In a brief overview, the method 100 may include providing a mycelium product comprising fungal mycelium, at 102. The method 100 may include exposing the mycelium product to a light so as to form an enhanced mycelium product having a vitamin D content, at 104.

In further detail, the method 100 may include providing a mycelium product comprising fungal mycelium, at 102. The initial vitamin D content of the mycelium product may be about 0 micrograms per 100 grams of the mycelium product. In some embodiments, the mycelium product may be a mycelium mass which may be fungal mycelium. The mycelium mass can include fungi from Ascomycota and Zygomycota, including the genera Aspergillus, Fusarium, Neurospora, and Monascus. Other species can include edible varieties of division Basidiomycota and generus Lentinula. One genus is Neurospora, which is used in food production through solid fermentation. The genus of Neurospora are known for highly efficient biomass production as well as ability to break down complex carbohydrates. For certain species of Neurospora, no known allergies have been detected and no levels of mycotoxins are produced. In some embodiments, the fungal mycelium comprises fungi from the genus Neurospora. In some embodiments, the fungal mycelium consists of fungi from the genus Neurospora. In some embodiments, the fungal mycelium comprises fungi from the species Neurospora crassa. In some embodiments, the fungal mycelium consists of fungi from the species Neurospora crassa. In addition to monocultures of filamentous fungi, multiple strains can be cultivated at once to tune the protein, amino acid, mineral, texture, and flavor profiles of the final biomass. The aforementioned species of fungi do not inherently have vitamin D.

Methods of growing fungal cells to produce mycelium are known in the art and include, as non-limiting examples, methods described in U.S. Patent Application Publication No. US 2019/0373935 A1 (entitled “METHOD FOR GROWING FUNGAL MYCELIUM AND FORMING EDIBLE PRODUCTS THEREFROM”), which is herein incorporated by reference, specifically in the passages reproduced below.

In one example, Neurospora crassa (N. crassa) was grown in batch configuration in a 10 L benchtop reactor. N. crassa is first grown on agar slants and incubated for 3 days at 32° C. Conidia or spores of the N. crassa are transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 2 g/L potassium phosphate monobasic, 1 g/L sodium nitrate, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 24 hours, the mycelium is harvested using a cheese cloth, dewatered in a cider press, and completely dried in a dehydrator set at 74° C. The total cell dry weight is 9.5 g/L. Protein analysis yields a crude protein content of 57 wt %. Amino acid analysis yields a PDCAAS score of 1.0 for the fibrous mycelium mass. The fibrous mycelium mass has a combined methionine and cysteine content of 26 mg/g crude protein.

In another example, N. crassa was grown in batch configuration in a 10 L benchtop reactor. N. crassa is first grown on agar slants and incubated for 3 days at 32° C. Conidia or spores of the N. crassa are transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 24 hours, the mycelium is harvested using a cheese cloth, dewatered in a cider press, and completely dried in a dehydrator set at 74° C. The total cell dry weight is 9 g/L. Protein analysis yields a crude protein content of 55 wt %. Amino acid analysis yields a PDCAAS score of 1.0 for the fibrous mycelium mass. The fibrous mycelium mass has a combined methionine and cysteine content of 26 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 30 g/L sucrose, 3 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 24 hours, the mycelium is harvested using a cheese cloth, dewatered in a cider press, and completely dried in a dehydrator set at 74° C. The total cell dry weight is 11 g/L. Protein analysis yields a crude protein content of 63 wt %. Amino acid analysis yields a PDCAAS score of 1.0 for the fibrous mycelium mass. The fibrous mycelium mass has a combined methionine and cysteine content of 27 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 3.25 g/L urea, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 24 hours, the mycelium is harvested using a cheese cloth, dewatered in a cider press, and completely dried in a dehydrator set at 74° C. The total cell dry weight is 8.5 g/L. Protein analysis yields a crude protein content of 56 wt %. Amino acid analysis yields a PDCAAS score of 1.0. The fibrous mycelium mass has a combined methionine and cysteine content of 25 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 15% ammonium hydroxide buffer. After 24 hours, the mycelium is harvested using a cheese cloth, dewatered in a cider press, and completely dried in a dehydrator set at 74° C. The total cell dry weight is 10 g/L. Protein analysis yields a crude protein content of 60 wt %. Amino acid analysis yields a PDCAAS score of 1.0. The fibrous mycelium mass has a combined methionine and cysteine content of 26 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 12 hours, 10 g/L sucrose and 1 g/L ammonium nitrate is added to the system. After 24 hours total, the mycelium is harvested using a cheese cloth, dewatered in a cider press, and completely dried in a dehydrator set at 74° C. The total cell dry weight is 12 g/L. Protein analysis yields a crude protein content of 60 wt %. Amino acid analysis yields a PDCAAS score of 1.0 for the fibrous mycelium mass. The fibrous mycelium mass has a combined methionine and cysteine content of 26 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 24 hours, 90% of the media is harvested; new media is added in the concentrations of above to bring the total system back to 10 L. The new sequential batch time is reduced to 12 hours. Every 12 hours 90% is harvested and the fed-batch process is repeated again. The process was carried out for 60 hours. The harvested cell dry weight is 9.5 g/L. Protein analysis yields a crude protein content of 60 wt %. Amino acid analysis yields a PDCAAS score of 1.0 for the fibrous mycelium mass. The fibrous mycelium mass has a combined methionine and cysteine content of 26 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 24 hours, 90% of the media is harvested; new media is added in the concentrations of above to bring the total system back to 10 L. The new sequential batch time is reduced to 12 hours. Every 12 hours 90% is harvested and the fed-batch process is repeated again. The process was carried out for 60 hours. Following straining with cheese cloth and pressing, all media is collected, autoclaved and reused by only adding 20 g/L sucrose, 2 g/L ammonium nitrate, and 1 g/L potassium phosphate monobasic. The repeated fed-batch process is carried out for 60 hours total. The harvested cell dry weight is 9.5 g/L. Protein analysis yields a crude protein content of 60 wt %. Amino acid analysis yields a PDCAAS score of 1.0 for the fibrous mycelium mass. The fibrous mycelium mass has a combined methionine and cysteine content of 26 mg/g crude protein.

The fungal cells may be grown to produce mycelium in a growth media which may include nutrients such as a sugar, a nitrogen-containing compound, and a phosphate-containing compound in a broth. The sugar can be in the range of about 5 to about 50 g/L (e.g., 5 g/L, 10 g/L, 20 g/L, 30 g/L, 40 g/L, or 50 g/L, inclusive). The sugar can include sucrose, glucose, fructose, molasses, or a mixture of sugars. The nitrogen-containing compound can be in the range of about 0.5 to about 10 g/L (e.g., 0.5 g/L, 1 g/L, 2 g/L, 3 g/L, 4 g/L, 5 g/L, or 10 g/L, inclusive). The nitrogen-containing compound can include ammonium hydroxide, ammonium nitrate, ammonium sulfate, ammonium chloride, urea, yeast extract, peptone, or a mixture of nitrogen-containing compounds. The phosphate-containing compound can be in the range of about 0.1 to about 5 g/L (e.g., 0.1 g/L, 0.2 g/L, 0.3 g/L, 0.4 g/L, 0.5 g/L, 1 g/L, 2 g/L, 3 g/L, 4 g/L, or 5 g/L, inclusive). The phosphate-containing compound can be potassium phosphate, sodium phosphate, phosphoric acid, or a mixture of phosphate-containing compounds.

The fungal cells can be grown at a temperature in a range of about 25° C. to about 45° C. (e.g., 25° C., 30° C., 35° C., 40° C., or 45° C., inclusive). The fungal cells can be grown in a range of about 12 hours to about 48 hours (e.g., 12 hours, 18 hours, 24 hours, 30 hours, 36 hours, 42 hours, or 48 hours, inclusive). Growing fungal cells can produce a yield of about 5 to about 20 g/L (e.g., 5 g/L, 10 g/L, 15 g/L, or 20 g/L, inclusive) of fungal cell dry weight. The mycelium mass can have a protein content of greater than or equal to about 40 wt % (e.g., 40 wt %, 50 wt %, 60 wt %, 70 wt %, 80 wt %, 90 wt %, or 100 wt %, inclusive) (dry weight). In some embodiments, the mycelium mass may have a protein content of about 50 to about 65% (e.g., 50%, 55%, 60%, or 65%, inclusive) (dry weight). The mycelium mass can have a combined methionine and cysteine content of at least about 25 mg/g crude protein.

In at least some embodiments of growing fungal cells disclosed herein (e.g., growing fungal cells such that the fungal cells produce a mycelium mass), the fungal cells are grown through a submerged fermentation process or in liquid media. Accordingly, embodiments of growing fungal cells (e.g., growing fungal cells such that the fungal cells produce a mycelium mass) described herein are distinguished from processes in which growth occurs on a substrate, such as in solid-state fermentation or surface fermentation.

Fungal cells grown through a submerged fermentation process can be grown either as aggregated masses referred to as pellets, as filamentous mycelium, or as a combination of both pellets and filamentous mycelium. In at least some embodiments of growing fungal cells disclosed herein (e.g., growing fungal cells such that the fungal cells produce a mycelium mass), the fungal cells grow as filamentous mycelium that is free of pellets.

The mycelium mass may then be separated from the growth media. In some embodiments, the mycelium mass may be mixed with food additives or colors. Additives can include vegetable or animal proteins, fats, emulsifiers, thickeners, stabilizers, flavoring, any other suitable additive, or combinations thereof.

In some embodiments, the mycelium product provided at 102 may be the mycelium mass during or after processing so as to where a mycelium product is formed. For example, any suitable process may be applied to the mycelium mass to form a mycelium product such as, but not limited to, dehydrating, rehydrating, compacting, pressing, texturizing, any combination thereof, or any other suitable process. In some embodiments, the mycelium product may be in a wet form, for example, have a moisture content of greater than about 90 wt % (e.g., 91 wt %, 92 wt %, 93 wt %, 94 wt %, 95 wt %, 96 wt %, 97 wt %, 98 wt %, or 99 wt %, inclusive). In other embodiments, the mycelium product may be compacted so as to have a moisture content of less than about 80 wt % (e.g. 79 wt %, 70 wt %, 60 wt %, 50 wt %, 40 wt %, 30 wt %, 20 wt %, 10 wt %, or 0 wt %, inclusive). In other embodiments, the mycelium product may be in a dehydrated, dry form, for example, have a moisture content of less than 10 wt % (e.g., 9 wt %, 8 wt %, 7 wt %, 6 wt %, 4 wt %, 4 wt %, 3 wt %, 2 wt %, 1 wt %, or 0 wt %, inclusive). In some embodiments, the mycelium product may be fully formed after processing. In some embodiments, the mycelium product may be partially formed and in the middle of processing.

The method 100 may include exposing the mycelium product to a light so as to form an enhanced mycelium product having a vitamin D content, at 104. In exposing the mycelium product to the light, a wavelength of the light, a distance to the light, and an exposure time may be varied. The light may have a wavelength in a range of about 100 to about 400 nanometers (e.g., 100 nanometers, 150 nanometers, 200 nanometers, 250 nanometers, 300 nanometers, 350 nanometers, or 400 nanometers, inclusive). The mycelium product may be exposed to the light for a first period of time. In some embodiments, the light may have a wavelength in the Ultraviolet (UV) radiation region. The UV radiation region may include three types of UV rays such as UVA, UVB, and UVC which all have different wavelengths. In some embodiments, the wavelength may be in the UVA region in a range of about 315 to about 400 nanometers (e.g., 315 nanometers, 325 nanometers, 335 nanometers, 345 nanometers, 355 nanometers, 365 nanometers, 375 nanometers, 385 nanometers, 395 nanometers, or 400 nanometers, inclusive). In some embodiments, the wavelength may be in the UVB region in a range of about 280 to about 315 nanometers (e.g., 280 nanometers, 290 nanometers, 300 nanometers, 310 nanometers, or 315 nanometers, inclusive). In some embodiments, the wavelength may be in the UVC region in a range of about 100 to about 280 nanometers (e.g., 100 nanometers, 120 nanometers, 140 nanometers, 160 nanometers, 180 nanometers, 200 nanometers, 220 nanometers, 240 nanometers, 260 nanometers, or 280 nanometers, inclusive). The light may have any suitable light source such as, but not limited to a UVA lightbulb, a UVB lightbulb, a UVC lightbulb or, a UV lamp. In some embodiments, the mycelium product may be exposed to light (e.g., UV light) while the mycelium product is disposed in a vessel (e.g., a fermentation tank), such as by positioning a source of light (e.g., UV light) within the vessel containing the mycelium product. In some embodiments, the mycelium product may be wet and exposed to UV radiation in a transparent conduit (e.g., a glass tube, a transparent container having an inlet and an outlet, etc.) as it is pumped from a tank (e.g., a fermentation tank) to processing (e.g., post-harvest processing). In other embodiments, the mycelium product may be exposed to UV light as it travels on a conveyer (e.g., a conveyor belt or chain) at any point of processing up until packaging. Thus, it is to be understood that a source of light (e.g., UV light) may be located in the same space as mycelium product (e.g., over or near a conveyer or within vessel containing mycelium product) or located externally to a vessel containing mycelium product (e.g., outside of a conduit carrying mycelium product).

The mycelium product is exposed to the light for a first period of time. In some embodiments, the first period of time is in a range of about 1 seconds to about 300 seconds (e.g., 1 second, 10 seconds, 20 seconds, 30 seconds, 40 seconds, 50 seconds, 60 seconds, 90 seconds, 120 seconds, 150 seconds, 180 seconds, 210 seconds, 240 seconds, 270 seconds, or 300 seconds, inclusive). In some embodiments, the first period of time is in a range of about 25 to about 45 seconds (e.g., 25 seconds, 30 seconds, 35 seconds, 40 seconds, or 45 seconds, inclusive). In some embodiments, the mycelium product is at a distance from a source of the light. In some embodiments, the distance is in a range of about 1 to about 10 centimeters (e.g., 1 centimeter, 2 centimeters, 3 centimeters, 4 centimeters, 5 centimeters, 6 centimeters, 7 centimeters, 8 centimeters, 9 centimeters, or 10 centimeters, inclusive). In some embodiments, the amount of a UV dose exposed to the mycelium product may be varied based on time and intensity of exposure. In such embodiments, the dose may be modeled by Equation 1.

$$U V Dose = U V Intensity [ \frac{μ W}{{cm}^{2}} ] * Exposure Time [ sec ] \qquad [ 1 ]$$

In some embodiments, exposing the mycelium product to the light comprises exposing a first side of the mycelium product to the light for the first period of time. In such embodiments, a second side of the mycelium product may be exposed to the light for a second period of time after the first time period. For example, the mycelium product may be flipped over to the second side (e.g., a back side, or a side orthogonal to the first side) of the mycelium product is exposed to the light, or moved laterally to align the second side (e.g., a second portion of a top side). The second period of time may be in a range of about 1 to about 300 seconds (e.g., 1 second, 10 seconds, 20 seconds, 30 seconds, 40 seconds, 50 seconds, 60 seconds, 90 seconds, 120 seconds, 150 seconds, 180 seconds, 210 seconds, 240 seconds, 270 seconds, or 300 seconds, inclusive). The second period of time may be in a range of about 25 to about 45 seconds (e.g., 25 seconds, 30 seconds, 35 seconds, 40 seconds, or 45 seconds, inclusive). The second period of time may be the same as the first period of time, or different than the first period of time. The second side may be on the same surface as the first side, or on a different surface from the first side. The second side may overlap the first side. The second side may have an area that is larger than, less than, or equal to an area of the first side.

The second side may be exposed to the light in the same wavelength range or region (e.g., UVA, UVB, or UVC) as the first side. The second side may be exposed to the light in a different wavelength range or different region as the first side. The second side may be exposed to light from the same source or a different source as the first side. The second side may be at a distance from the source of light in a range of about 1 to about 10 centimeters (1 centimeters, 2 centimeters, 3 centimeters, 4 centimeters, 5 centimeters, 6 centimeters, 7 centimeters, 8 centimeters, 9 centimeters, or 10 centimeters, inclusive). The second side may be at the same distance or a different distance from the source of light as the first side.

Exposing the mycelium product to the light (e.g., UV radiation) may cause intracellular production of vitamin D in the mycelium product so as to produce an enhance mycelium product with a vitamin D content. Thus, unnatural fortification of the mycelium product by separately adding vitamin D thereto, is not required. The mycelium product may contain ergosterol, a vitamin D precursor, which may be converted to vitamin D₂ by exposure to UV light.

The vitamin D content of the mycelium product after exposure to the light (e.g., UV light) may be greater than or equal to about 1 microgram per 100 grams of the enhanced mycelium product (e.g., 1 microgram per 100 grams, 2 micrograms per 100 grams, 3 micrograms per 100 grams, 4 micrograms per 100 grams, 5 micrograms per 100 grams, etc.).

In some embodiments, the vitamin D content may meet or exceed the RDA for vitamin D in the enhanced mycelium product. The RDA for vitamin D for children 1 to 18 years old, inclusive, may be 15 micrograms. The RDA for vitamin D for adults 19 to 70 years old, inclusive, may be 15 micrograms. The RDA for vitamin D for adults 71 years and older may be 20 micrograms. The RDA for vitamin D for pregnant and breastfeeding women may be 15 micrograms. Thus, in some embodiments, the vitamin D content of the mycelium product after exposure to the light (e.g., UV light) may be about 15 micrograms per 100 g of enhanced mycelium product or about 20 micrograms per 100 g of enhanced mycelium product. In some embodiments, the vitamin D content of the mycelium product after exposure to the light (e.g., UV light) may be greater than about 15 micrograms per 100 g of enhanced mycelium product or greater than about 20 micrograms per 100 g of enhanced mycelium product.

In some embodiments, the vitamin D content of the mycelium product after exposure to the light (e.g., UV light) does not exceed the tolerable daily upper intake level of vitamin D. The tolerable upper intake level of vitamin D for individuals aged 9 years and older may be 100 micrograms. Thus, in some embodiments, the vitamin D content of the mycelium product after exposure to the light (e.g., UV light) may be about 100 micrograms or less per 100 g of enhanced mycelium product.

In some embodiments, the vitamin D content of the mycelium product after exposure to the light (e.g., UV light) may be from about 1 microgram per 100 grams of the enhanced mycelium product to about 1 milligram (i.e., 1,000 micrograms) per 100 grams of the enhanced mycelium product. For example, in some embodiments, the vitamin D content of the mycelium product after exposure to the light (e.g., UV light) may be from about 1 microgram per 100 grams of the enhanced mycelium product to about 100 micrograms per 100 grams of the enhanced mycelium product. In some embodiments, the vitamin D content of the mycelium product after exposure to the light (e.g., UV light) may be from about 1 microgram per 100 grams of the enhanced mycelium product to about 200 micrograms per 100 grams of the enhanced mycelium product. In some embodiments, the vitamin D content of the mycelium product after exposure to the light (e.g., UV light) may be from about 1 microgram per 100 grams of the enhanced mycelium product to about 300 micrograms per 100 grams of the enhanced mycelium product. In some embodiments, the vitamin D content of the mycelium product after exposure to the light (e.g., UV light) may be from about 1 microgram per 100 grams of the enhanced mycelium product to about 400 micrograms per 100 grams of the enhanced mycelium product. In some embodiments, the vitamin D content of the mycelium product after exposure to the light (e.g., UV light) may be from about 1 microgram per 100 grams of the enhanced mycelium product to about 500 micrograms per 100 grams of the enhanced mycelium product. In some embodiments, the vitamin D content of the mycelium product after exposure to the light (e.g., UV light) may be from about 1 microgram per 100 grams of the enhanced mycelium product to about 600 micrograms per 100 grams of the enhanced mycelium product. In some embodiments, the vitamin D content of the mycelium product after exposure to the light (e.g., UV light) may be from about 1 microgram per 100 grams of the enhanced mycelium product to about 700 micrograms per 100 grams of the enhanced mycelium product. In some embodiments, the vitamin D content of the mycelium product after exposure to the light (e.g., UV light) may be from about 1 microgram per 100 grams of the enhanced mycelium product to about 800 micrograms per 100 grams of the enhanced mycelium product. In some embodiments, the vitamin D content of the mycelium product after exposure to the light (e.g., UV light) may be from about 1 microgram per 100 grams of the enhanced mycelium product to about 900 micrograms per 100 grams of the enhanced mycelium product. In some embodiments, the vitamin D content of the mycelium product after exposure to the light (e.g., UV light) may be from about 1 microgram per 100 grams of the enhanced mycelium product to about 5 micrograms per 100 grams of the enhanced mycelium product, from about 1 microgram per 100 grams of the enhanced mycelium product to about 10 micrograms per 100 grams of the enhanced mycelium product, from about 1 microgram per 100 grams of the enhanced mycelium product to about 15 micrograms per 100 grams of the enhanced mycelium product, or from about 1 microgram per 100 grams of the enhanced mycelium product to about 20 micrograms per 100 grams of the enhanced mycelium product, such as about 1 microgram per 100 grams of the enhanced mycelium product, about 2 micrograms per 100 grams of the enhanced mycelium product, about 3 microgram per 100 grams of the enhanced mycelium product, about 4 micrograms per 100 grams of the enhanced mycelium product, about 5 micrograms per 100 grams of the enhanced mycelium product, about 6 micrograms per 100 grams of the enhanced mycelium product, about 7 micrograms per 100 grams of the enhanced mycelium product, about 8 micrograms per 100 grams of the enhanced mycelium product, about 9 micrograms per 100 grams of the enhanced mycelium product, about 10 micrograms per 100 grams of the enhanced mycelium product, about 11 micrograms per 100 grams of the enhanced mycelium product, about 12 micrograms per 100 grams of the enhanced mycelium product, about 13 micrograms per 100 grams of the enhanced mycelium product, about 14 micrograms per 100 grams of the enhanced mycelium product, about 15 micrograms per 100 grams of the enhanced mycelium product, about 16 micrograms per 100 grams of the enhanced mycelium product, about 17 micrograms per 100 grams of the enhanced mycelium product, about 18 micrograms per 100 grams of the enhanced mycelium product, about 19 micrograms per 100 grams of the enhanced mycelium product, or about 20 micrograms per 100 grams of the enhanced mycelium product.

In some embodiments, the mycelium product may be exposed to the UV light for a period of time outside of the range of 1 to 300 seconds. In such embodiments, the vitamin D content may be greater than about 100 micrograms per 100 grams of the enhanced mycelium product (e.g., greater than 1,000 micrograms per 100 grams of the enhanced mycelium product.) In such embodiments, an excessive amount of vitamin D may be toxic to humans, for examples, who are mildly vitamin D sufficient. However, such high vitamin D content enhanced mycelium may be used to produce mycelium based edible meat substitutes for animals or severely deficient humans who need a high dose of vitamin D so as to increase their blood vitamin D levels to a normal range in a short amount of time. Such high vitamin D content enhanced mycelium may also be used to produce a vitamin D supplement.

In some embodiments, exposure of the mycelium product to light (e.g., UV light) may induce intracellular production of vitamin D in the mycelium product to such an extent so as to produce an enhanced mycelium product with a vitamin D content that renders the enhanced mycelium product unfit for human consumption (for example, because its vitamin D content is so high as to create a risk of vitamin D toxicity if consumed by humans). The present disclosure provides approaches to address that concern. In some embodiments, the vitamin D content in an enhanced mycelium product is controlled (e.g., to ensure vitamin D levels that are appropriate for human consumption) by moderating the duration of exposure of the mycelium product to light (e.g., UV light) and/or by moderating the wavelength and/or intensity of the light source. In some embodiments, the duration of exposure of the mycelium product to light (e.g., UV light) is moderated by adjusting and/or controlling the flow rate of a fluid phase containing mycelium product as it is exposed to light (e.g., UV light). In other embodiments, only a portion of a quantity of mycelium product is exposed to light (e.g., UV light) that induces intracellular production of vitamin D in the mycelium product so as to produce an enhanced mycelium product, and that portion is mixed with additional amounts of non-irradiated mycelium product. In some embodiments, the admixture of a portion of mycelium product exposed to light (e.g., UV light) and an amount of non-irradiated mycelium product has, as a whole, a vitamin D content that is low enough to be suitable for human consumption. For example, in some embodiments, such an admixture has a vitamin D content from about 1 microgram per 100 grams of the enhanced mycelium product to about 20 micrograms per 100 grams of the enhanced mycelium product, such as from about 1 microgram per 100 grams of the enhanced mycelium product to about 10 micrograms per 100 grams of the enhanced mycelium product or from about 1 microgram per 100 grams of the enhanced mycelium product to about 15 micrograms per 100 grams of the enhanced mycelium product.

In some embodiments where only a portion of a quantity of mycelium product is exposed to light (e.g., UV light) that induces intracellular production of vitamin D in the mycelium product so as to produce an enhanced mycelium product, that portion of the quantity of mycelium product is in a fluid phase and is routed to an apparatus containing a light source (e.g., a UV light source, such as a UVB light source), exposed to light (e.g., UV light), and combined with remaining non-irradiated mycelium product in a fluid phase. For example, in an embodiment, a portion of a fluid phase containing mycelium is routed from a fermentation tank or other vessel to an apparatus containing a light source (e.g., a UV light source, such as a UVB light source), exposed to light, and recombined with non-irradiated mycelium product in a fluid phase. In some such embodiments, the irradiated portion of the fluid phase is recombined with non-irradiated mycelium product through a static mixture to provide a uniform dispersion of irradiated and non-irradiated mycelium product. In some embodiments, the apparatus containing a light source is a modified version of a device used for sanitizing water with light (e.g., UV light), such as a Sanitron® device. In some embodiments, the amount of fluid-phase mycelium product that is routed to the apparatus and exposed to light (e.g., UV light) is from about 1/40^{th} to about 1/10^{th} of the total amount of mycelium product prepared, such as about 1/40^{th}, 1/35^{th}, 1/30^{th}, 1/25^{th}, 1/20^{th}, 1/15^{th}, or 1/10^{th} of the total amount of mycelium product prepared. For example, in an embodiment where the amount of fluid-phase mycelium product that is routed to the apparatus and exposed to light (e.g., UV light) is 1/10^{th} of the total amount of mycelium product prepared, it is to be understood that 1/10^{th} of the entire volume of fluid phase mycelium product is routed to the apparatus and exposed to light (e.g., UV light), with 9/10^{th} of the entire volume of fluid phase mycelium product remaining non-irradiated. The 1/10^{th} of the entire volume of fluid phase mycelium product that was exposed to light (e.g., UV light) is recombined with the remaining 9/10^{th} of the entire volume of fluid phase mycelium product, providing a volume of fluid phase mycelium product that, as a whole, has a higher vitamin D content compared to the vitamin D content of the volume of fluid phase mycelium product prior to irradiation of the portion of fluid phase mycelium product. In some embodiments, the light source in the apparatus is a UVB light source. In some embodiments, the light source in the apparatus is a UVB light source emitting UVB light having a wavelength that is from about 280 nanometers to about 315 nanometers.

In some embodiments, food additives or colors may be added to the enhanced mycelium product or the mycelium product at any step in the method 100. Additives can include vegetable or animal proteins, fats, emulsifiers, thickeners, stabilizers, flavoring, any other suitable additive, or combinations thereof. The colors can include any suitable color additive such as liquids, powders, gels, and pastes and may provide a similar color to meat products. The food additives or colors may be uniformly distributed in the mycelium product.

In some embodiments, a flavorant may be added to the enhanced mycelium product. The flavorant can include flavorings or food additives. For example, the flavorant can include an oil, such as a nut-derived oil, vegetable-derived oil, plant-derived oil, and animal-derived oil. The flavorant can include spices (e.g., black pepper, fennel, mustard, nutmeg, cinnamon, ginger, cayenne pepper, clove, etc.). The flavorant can include a flavored powder (e.g., onion powder, garlic powder, BBQ powder, sour cream powder, lemon powder, lime powder, etc.).

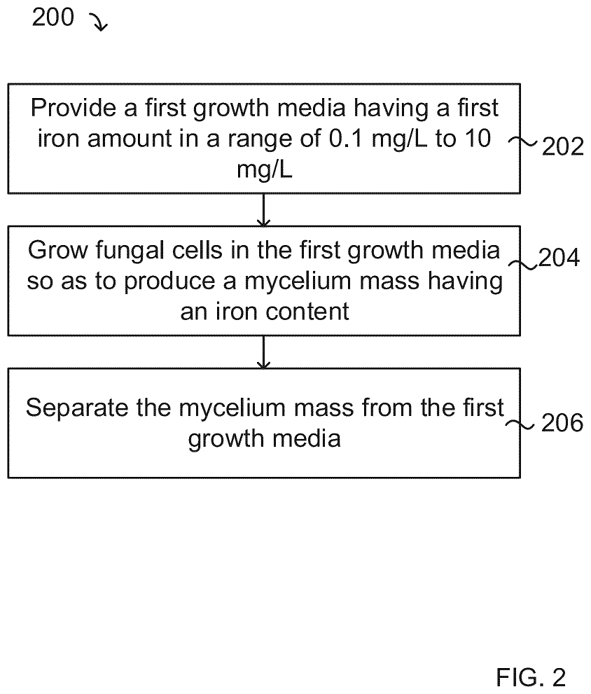

FIG. 2 illustrates a block diagram of an example method 200 for forming an edible meat substitute product, according to an embodiment. The example method 200 may increase the iron content of a mycelium mass. In a brief overview, the method 200 may include providing a first growth media having a first iron amount in a range of 0.1 mg/L to 10 mg/L, at 202. The method 200 may include growing fungal cells in the first growth media so as to produce a mycelium mass having an iron content, at 204. The method 200 may include separating the mycelium mass from the first growth media, at 206.

In further detail, the method 200 may include providing a first growth media having a first iron amount in a range of 0.1 mg/L to 10 mg/L, at 202. The first growth media may be have a first iron amount in a range of about 0.1 to about 10 mg/L (e.g., 0.1 mg/L, 1 mg/L, 2 mg/L, 3 mg/L, 4 mg/L, 5 mg/L, 6 mg/L, 7 mg/L, 8 mg/L, 9 mg/L, or 10 mg/L, inclusive). In some embodiments, the first iron amount is in a range of about 0.1 to 2 mg/L (e.g., 0.1 mg/L, 0.2 mg/L, 0.3 mg/L, 0.4 mg/L, 0.5 mg/L, 0.6 mg/L, 0.7 mg/L, 0.8 mg/L, 0.9 mg/L, 1 mg/L, 1.1 mg/L, 1.2 mg/L, 1.3 mg/L, 1.4 mg/L, 1.5 mg/L, 1.6 mg/L, 1.7 mg/L, 1.8 mg/L, 1.9 mg/L, or 2 mg/L, inclusive). The first iron amount may be adjusted according to the desired iron content. The first iron amount may be provided by any suitable iron-containing material, for example, iron salts (e.g., ferric citrate, ferrous sulfate, ferrous gluconate, ferrous fumarate, ferric sulfate, ammonium ion sulfate, heme iron polypeptides, carbonyl iron, etc.)

The first growth media may be substantially similar to the growth media discussed in the method 100. The first growth media may be contained in a vessel, such as a vat capable of growing several kilograms of fungal mycelium. The first growth media may include nutrients such as sugar, a nitrogen-containing compound, and a phosphate-containing compound in a broth or these nutrients may be added in. The sugar can be in the range of about 5 to about 50 g/L (e.g., 5 g/L, 10 g/L, 20 g/L, 30 g/L, 40 g/L, or 50 g/L, inclusive). The sugar can include sucrose, glucose, fructose, molasses, or a mixture of sugars. The nitrogen-containing compound can be in a range of about 0.5 to about 10 g/L (e.g., 0.5 g/L, 1 g/L, 2 g/L, 3 g/L, 4 g/L, 5 g/L, or 10 g/L, inclusive). The nitrogen-containing compound can include ammonium hydroxide, ammonium nitrate, ammonium sulfate, ammonium chloride, urea, yeast extract, peptone, or a mixture of nitrogen-containing compounds. The phosphate-containing compound can be in the range of about 0.1 to about 5 g/L (e.g., 0.1 g/L, 0.2 g/L, 0.3 g/L, 0.4 g/L, 0.5 g/L, 1 g/L, 2 g/L, 3 g/L, 4 g/L, or 5 g/L, inclusive). The phosphate-containing compound can be potassium phosphate, sodium phosphate, phosphoric acid, or a mixture of phosphate-containing compounds.

The method 200 may include growing fungal cells in the first growth media such that the fungal cells (e.g., those described with respect to the method 100) produce a mycelium mass having an iron content, at 204. The iron content may be greater than or equal to 5 mg of iron per 100 g of mycelium mass. In some embodiments, the mycelium mass has an iron content of from about 5 mg of iron per 100 g of mycelium mass to about 15 mg of iron per 100 g of mycelium mass. The fungal cells can be grown at a temperature in a range of about 25° C. to about 45° C. (e.g., 25° C., 30° C., 35° C., 40° C., or 45° C., inclusive). The fungal cells can be grown in a range of about 12 hours to about 48 hours (e.g., 12 hours, 18 hours, 24 hours, 30 hours, 36 hours, 42 hours, or 48 hours, inclusive). Growing fungal cells can produce a yield of about 5 to about 20 g/L (e.g., 5 g/L, 10 g/L, 15 g/L, or 20 g/L, inclusive) of fungal cell dry weight. The mycelium mass can have a protein content of greater than or equal to about 40 wt % (e.g., 40 wt %, 50 wt %, 60 wt %, 70 wt %, 80 wt %, 90 wt %, or 100 wt %, inclusive) (dry weight). In some embodiments, the mycelium mass may have a protein content of about 50 to about 65% (e.g., 50%, 55%, 60%, or 65%, inclusive) (dry weight). The mycelium mass can have a combined methionine and cysteine content of at least about 25 mg/g crude protein. In some embodiments, nutrients such as the sugar, the nitrogen-containing compound, or the phosphate-containing compound are added to the first growth media after the fungal cells are grown.

Growing the mycelium mass in first growth media with an increased iron content may allow the mycelium mass to accumulate the iron intracellularly so as to boost the iron content of the mycelium mass without fortification with added salts or additives after the final product has been formed. In some embodiments, the mycelium mass may meet or exceed the RDA for iron, which is 8 milligrams/day for men, for women aged 19-50 years is 18 milligrams/day, and for women aged 51 and older is 8 milligrams/day. In some embodiments, increasing the first iron amount in the first growth media will increase the iron content of the mycelium mass without impacting growth rate.

The method 200 may include separating the mycelium mass from the first growth media, at 206. Separating the mycelium mass from the first growth media can be performed using gravity straining, centrifugation, a belt press, a filter press, a mechanical press, a drum dryer, or any other suitable process. The separated mycelium mass can have a moisture content of greater than about 90 wt %, (e.g., 91 wt %, 92 wt %, 93 wt %, 94 wt %, 95 wt %, 96 wt %, 97 wt %, 98 wt %, or 99 wt %, inclusive). During the separation process, the mycelium mass can be washed with water, ethanol, acid, base or other solvent. Recovered filtrate can be reused or discarded. Cell walls of the mycelium mass can be disrupted, for example, through lysing. Lysis may be performed by adjusting the pH to below 4 or above 9, by adding lysis enzymes, by raising the temperature in a range of 40° C. and 60° C. in a range of 1 and 24 hours, or any other suitable lysis method. Following separation, additives (e.g., food additives) can be mixed with the mycelium mass. Additives can include vegetable or animal proteins, fats, emulsifiers, thickeners, stabilizers, and flavoring, for example, when the mycelium mass is being formed into an edible product.

In some embodiments, a second growth media may be added to the mycelium mass with a second iron amount in a range of about 0.1 to about 10 mg/L (e.g., 0.1 mg/L, 1 mg/L, 2 mg/L, 3 mg/L, 4 mg/L, 5 mg/L, 6 mg/L, 7 mg/L, 8 mg/L, 9 mg/L, or 10 mg/L, inclusive). In some embodiments, the second iron amount is in a range of about 0.1 to 2 mg/L (e.g., 0.1 mg/L, 0.2 mg/L, 0.3 mg/L, 0.4 mg/L, 0.5 mg/L, 0.6 mg/L, 0.7 mg/L, 0.8 mg/L, 0.9 mg/L, 1 mg/L, 1.1 mg/L, 1.2 mg/L, 1.3 mg/L, 1.4 mg/L, 1.5 mg/L, 1.6 mg/L, 1.7 mg/L, 1.8 mg/L, 1.9 mg/L, or 2 mg/L, inclusive). The second iron amount may be the same as or different from the first iron amount. In some embodiments, the mycelium mass is in the first growth media for a time period greater than or equal to about 5 hours (e.g., 5 hours, 6 hours, etc.) and the second growth media for a time period greater than or equal to 30 seconds (e.g., 30 seconds, 60 seconds, 120 seconds, etc.).

In some embodiments, the second growth media may be provided to the mycelium mass after the complete removal and separation of the first growth media from the mycelium mass. In some embodiments, the second growth media may be provided after only a partial removal and separation of the first growth media from the mycelium mass. In some embodiments, the second growth media may be provided to the first growth media without removal of the first growth media from the mycelium mass. The amount of the second growth media may be equal to, less than, or greater than the original amount of the first growth media. In some embodiments, adding the second growth media may add more nutrients (e.g., iron) to the mycelium mass. Increasing the amount of iron in the second growth media or the first growth media may increase the iron content in the mycelium mass. In some embodiments, the second growth media may be added at various stages in the formation of the mycelium mass growth such as to a fermentation broth. It is understood that the number of growth media used to grow the mycelium mass is not limited to two, and any suitable number of growth media used to deliver nutrients (e.g., iron) may be used.

In some embodiments, an edible meat substitute product with an enhanced nutritional content may be formed by a process of providing a first growth media having a first iron amount in a range of 0.1 mg/L to 10 mg/L and growing fungal cells in the first growth media such that the fungal cells produce a mycelium mass. The mycelium mass may have an iron content greater than or equal to 5 mg of iron per 100 g of the mycelium mass. In some embodiments, the mycelium mass has an iron content of from about 5 mg of iron per 100 g of mycelium mass to about 15 mg of iron per 100 g of mycelium mass. The process may further include separating the mycelium mass from the first growth media. In some embodiments, the edible meat substitute product is formed by the method 200. The enhanced edible meat substitute product may have a higher iron content than an edible meat substitute product that does not undergo the process of method 200.

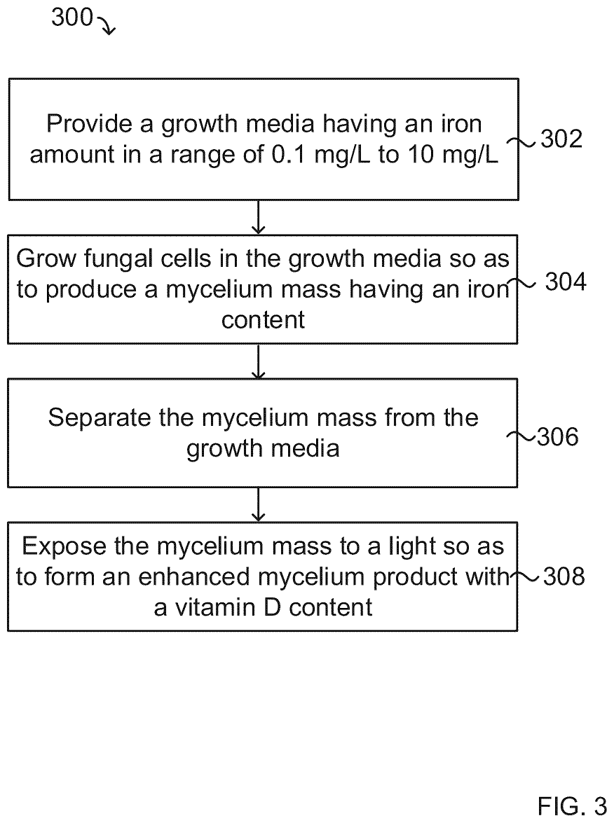

FIG. 3 illustrates a block diagram of an example method 300 for forming an edible meat substitute product, according to an embodiment. In particular, the example method 300 may increase both the vitamin D content and the iron content of an enhanced mycelium product. In a brief overview, the method 300 may include providing a growth media having an iron amount in a range of 0.1 mg/L to 10 mg/L, at 302. The method 300 may include growing fungal cells in the growth media so as to produce a mycelium mass having an iron content, at 304. The method 300 may include separating the mycelium mass from the growth media, at 306. The method 300 may include exposing the mycelium mass to a light so as to form an enhanced mycelium product with a vitamin D content, at 308

In further detail, the method 300 may include providing a first growth media having a first iron amount in a range of 0.1 mg/L to 10 mg/L, at 302. The first growth media may be substantially similar to the first growth media in the method 200. The first growth media may be have a first iron amount in a range of about 0.1 to about 10 mg/L (e.g., 0.1 mg/L, 1 mg/L, 2 mg/L, 3 mg/L, 4 mg/L, 5 mg/L, 6 mg/L, 7 mg/L, 8 mg/L, 9 mg/L, or 10 mg/L, inclusive). In some embodiments, the first iron amount is in a range of about 0.1 to 2 mg/L (e.g., 0.1 mg/L, 0.2 mg/L, 0.3 mg/L, 0.4 mg/L, 0.5 mg/L, 0.6 mg/L, 0.7 mg/L, 0.8 mg/L, 0.9 mg/L, 1 mg/L, 1.1 mg/L, 1.2 mg/L, 1.3 mg/L, 1.4 mg/L, 1.5 mg/L, 1.6 mg/L, 1.7 mg/L, 1.8 mg/L, 1.9 mg/L, or 2 mg/L, inclusive). The first iron amount may be adjusted according to the desired iron content. The first iron amount may be provided by any suitable iron-containing material such, but not limited to, iron salt.

The first growth media may be contained in a vessel, such as a vat capable of growing several kilograms of fungal mycelium. The first growth media may include nutrients such as sugar, a nitrogen-containing compound, and a phosphate-containing compound in a broth or these nutrients may be added in. The sugar can be in the range of about 5 to about 50 g/L (e.g., 5 g/L, 10 g/L, 20 g/L, 30 g/L, 40 g/L, or 50 g/L, inclusive). The sugar can include sucrose, glucose, fructose, molasses, or a mixture of sugars. The nitrogen-containing compound can be in the range of about 0.5 to about 10 g/L (e.g., 0.5 g/L, 1 g/L, 2 g/L, 3 g/L, 4 g/L, 5 g/L, or 10 g/L, inclusive). The nitrogen-containing compound can include ammonium hydroxide, ammonium nitrate, ammonium sulfate, ammonium chloride, urea, yeast extract, peptone, or a mixture of nitrogen-containing compounds. The phosphate-containing compound can be in the range of about 0.1 to about 5 g/L (e.g., 0.1 g/L, 0.2 g/L, 0.3 g/L, 0.4 g/L, 0.5 g/L, 1 g/L, 2 g/L, 3 g/L, 4 g/L, or 5 g/L, inclusive). The phosphate-containing compound can be potassium phosphate, sodium phosphate, phosphoric acid, or a mixture of phosphate-containing compounds.

The method 300 may include growing fungal cells in the first growth media such that the fungal cells (similar to those in the method 100) produce a mycelium mass having an iron content, at 304. The mycelium mass may have an iron content greater than or equal to about 5 mg of iron per 100 g of the mycelium mass. In some embodiments, the mycelium mass has an iron content of from about 5 mg of iron per 100 g of mycelium mass to about 15 mg of iron per 100 g of mycelium mass. The fungal cells can be grown at a temperature in a range of about 25° C. to about 45° C. (e.g., 25° C., 30° C., 35° C., 40° C., or 45° C., inclusive). The fungal cells can be grown in a range of about 12 hours to about 48 hours (e.g., 12 hours, 18 hours, 24 hours, 30 hours, 36 hours, 42 hours, or 48 hours, inclusive). Growing fungal cells can produce a yield of about 5 to about 20 g/L (e.g., 5 g/L, 10 g/L, 15 g/L, or 20 g/L, inclusive) of fungal cell dry weight. The mycelium mass can have a protein content of greater than or equal to about 40 wt % (e.g., 40 wt %, 50 wt %, 60 wt %, 70 wt %, 80 wt %, 90 wt %, or 100 wt %, inclusive) (dry weight). In some embodiments, the mycelium mass may have a protein content of about 50 to about 65% (e.g., 50%, 55%, 60%, or 65%, inclusive) (dry weight). The mycelium mass can have a combined methionine and cysteine content of at least about 25 mg/g crude protein. In some embodiments, nutrients such as the sugar, the nitrogen-containing compound, or the phosphate-containing compound are added to the first growth media after the fungal cells are grown.

The method 300 may include separating the mycelium mass from the first growth media, at 306. Separating the mycelium mass from the first growth media can be performed using gravity straining, centrifugation, a belt press, a filter press, a mechanical press, a drum dryer, or any other suitable process. The separated mycelium mass can have a moisture content of greater than about 90 wt %, (e.g., 91 wt %, 92 wt %, 93 wt %, 94 wt %, 95 wt %, 96 wt %, 97 wt %, 98 wt %, or 99 wt %, inclusive). During the separation process, the mycelium mass can be washed with water, ethanol, acid, base or other solvent. Recovered filtrate can be reused or discarded. Cell walls of the mycelium mass can be disrupted, for example, through lysing. Lysis may be performed by adjusting the pH to below 4 or above 9, by adding lysis enzymes, by raising the temperature in a range of 40° C. and 60° C. in a range of 1 and 24 hours, or any other suitable lysis method. Following separation, additives (e.g., food additives) can be mixed with the mycelium mass. Additives can include vegetable or animal proteins, fats, emulsifiers, thickeners, stabilizers, and flavoring, for example, when the mycelium mass is being formed into an edible product.

In some embodiments, a second growth media may be provided to the mycelium mass with a second iron amount in a range of about 0.1 to about 10 mg/L (e.g., 0.1 mg/L, 1 mg/L, 2 mg/L, 3 mg/L, 4 mg/L, 5 mg/L, 6 mg/L, 7 mg/L, 8 mg/L, 9 mg/L, or 10 mg/L, inclusive). In some embodiments, the second iron amount is in a range of about 0.1 to 2 mg/L (e.g., 0.1 mg/L, 0.2 mg/L, 0.3 mg/L, 0.4 mg/L, 0.5 mg/L, 0.6 mg/L, 0.7 mg/L, 0.8 mg/L, 0.9 mg/L, 1 mg/L, 1.1 mg/L, 1.2 mg/L, 1.3 mg/L, 1.4 mg/L, 1.5 mg/L, 1.6 mg/L, 1.7 mg/L, 1.8 mg/L, 1.9 mg/L, or 2 mg/L, inclusive). The second iron amount may be the same as or different from the first iron amount. In some embodiments, the mycelium mass is in the first growth media for a time period greater than or equal to about 5 hours (e.g., 5 hours, 6 hours, etc.) and the second growth media for a time period greater than or equal to 30 seconds (e.g., 30 seconds, 60 seconds, 120 seconds, etc.).

In some embodiments, the second growth media may be provided to the mycelium mass after the complete removal and separation of the first growth media from the mycelium mass. In some embodiments, the second growth media may be provided after only a partial removal and separation of the first growth media from the mycelium mass. In some embodiments, the second growth media may be provided to the first growth media without any removal of the first growth media from the mycelium mass. The amount of the second growth media may be equal to, less than, or greater than the original amount of the first growth media. In some embodiments, adding the second growth media may add more nutrients (e.g., iron) to the mycelium mass. Increasing the amount of iron in the second growth media or the first growth media may increase the iron content in the mycelium mass. In some embodiments, the second growth media may be added at various stages in the formation of the mycelium mass growth such as to a fermentation broth. It is understood that the number of growth media used to grow the mycelium mass is not limited to two, and any suitable number of growth media used to deliver nutrients (e.g., iron) may be used.

The method 300 may include exposing the mycelium mass to a light so as to form an enhanced mycelium product with a vitamin D content, at 308. The light may have a wavelength in a range of about 100 to about 400 nanometers (e.g., 100 nanometers, 150 nanometers, 200 nanometers, 250 nanometers, 300 nanometers, 350 nanometers, or 400 nanometers, inclusive), and the mycelium product may be exposed for a first period of time. In some embodiments, the light may have a wavelength in the Ultraviolet (UV) radiation region, for example, any suitable wavelength, as previously described herein. The light may have any suitable light source such as, but not limited to a UVA lightbulb, a UVB lightbulb, a UVC lightbulb or, a UV lamp.

The mycelium mass is exposed to the light for a first period of time. In some embodiments, the first period of time is in a range of about 1 to about 300 seconds (e.g., 1 second, 10 seconds, 20 seconds, 30 seconds, 40 seconds, 50 seconds, 60 seconds, 90 seconds, 120 seconds, 150 seconds, 180 seconds, 210 seconds, 240 seconds, 270 seconds, or 300 seconds, inclusive). In some embodiments, the first period of time is in a range of about 25 to about 45 seconds (e.g., 25 seconds, 30 seconds, 35 seconds, 40 seconds, or 45 seconds, inclusive). In some embodiments, the mycelium mass is at a distance from a source of the light. In some embodiments, the distance is in a range of about 1 to about 10 centimeters (e.g., 1 centimeter, 2 centimeters, 3 centimeters, 4 centimeters, 5 centimeters, 6 centimeters, 7 centimeters, 8 centimeters, 9 centimeters, or 10 centimeters, inclusive). In some embodiments, the amount of a UV dose exposed to the mycelium mass may be varied based on time and intensity of exposure. In such embodiments, the dose may be modeled by Equation 1.

In some embodiments, exposing the mycelium mass to the light includes exposing a first side of the mycelium mass to the light. In such embodiments, a second side of the mycelium mass is exposed to the light for a second period of time. The second period of time may be in a range of about 1 to about 300 seconds (e.g., 1 second, 10 seconds, 20 seconds, 30 seconds, 40 seconds, 50 seconds, 60 seconds, 90 seconds, 120 seconds, 150 seconds, 180 seconds, 210 seconds, 240 seconds, 270 seconds, or 300 seconds, inclusive). The second period of time may be in a range of about 25 to about 45 seconds (e.g., 25 seconds, 30 seconds, 35 seconds, 40 seconds, or 45 seconds, inclusive). The second period of time may be the same as the as the first period of time. The second period of time may be different than the first period of time. The second side may be the same as the first side. The second side may be different from the first side. The second side may overlap the first side. The second side may have an area that is larger than, less than, or equal to an area of the first side.

The second side may be exposed to the light in the same wavelength or region (e.g., UVA, UVB, or UVC) as the first side. The second side may be exposed to the light in a different wavelength or different region as the first side. The second side may be exposed to light from the same source or a different source as the first side. The second side may be at a distance from the source of light in a range of about 1 to about 10 centimeters (1 centimeters, 2 centimeters, 3 centimeters, 4 centimeters, 5 centimeters, 6 centimeters, 7 centimeters, 8 centimeters, 9 centimeters, or 10 centimeters, inclusive). The second side may be at the same distance or a different distance from the source of light as the first side.

Growing the mycelium mass in a growth media including iron, and exposing the mycelium product to UV light produces an enhanced mycelium product that has an iron content that may be greater than or equal to 5 mg per 100 gram of mycelium mass, and a vitamin D content that may be in a range of greater than about 1 microgram per 100 grams of the enhanced mycelium product. In some embodiments, the mycelium mass has an iron content of from about 5 mg of iron per 100 g of mycelium mass to about 15 mg of iron per 100 g of mycelium mass and a vitamin D content that may be in a range of greater than about 1 microgram per 100 grams of the enhanced mycelium product.

In some embodiments, the mycelium product may be exposed to the UV light for a period of time outside of the range of 1 to 300 seconds. In such embodiments, the vitamin D content may be greater than about 100 micrograms per 100 grams of the enhanced mycelium product (e.g., greater than 1,000 micrograms per 100 grams of the enhanced mycelium product). In such embodiments, an excessive amount of vitamin D may be toxic to humans, for example who are mildly vitamin D sufficient. However, such high vitamin D content enhanced mycelium may be used to produce mycelium based edible meat substitute for animals or severely deficient humans who need a high dose of vitamin D so as to increase their blood vitamin D levels to a normal range in a short amount of time. Such high vitamin D content enhanced mycelium may also be used to produce a vitamin D supplement.

In some embodiments, a flavorant may be added to the enhanced mycelium product. The flavorant can include flavorings or food additives. For example, the flavorant can include an oil, such as a nut-derived oil, vegetable-derived oil, plant-derived oil, and animal-derived oil. The flavorant can include spices (e.g., black pepper, fennel, mustard, nutmeg, cinnamon, ginger, cayenne pepper, clove, etc.). The flavorant can include a flavored powder (e.g., onion powder, garlic powder, BBQ powder, sour cream powder, lemon powder, lime powder, etc.).

Following are some examples of enhancing the nutritional content of mycelium products. These examples are for illustrative purposes only and should not be construed as limiting the disclosure in any shape or form.

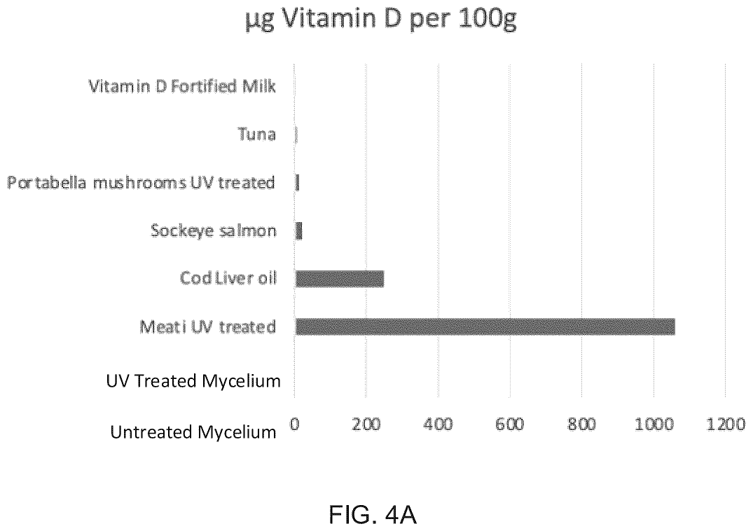

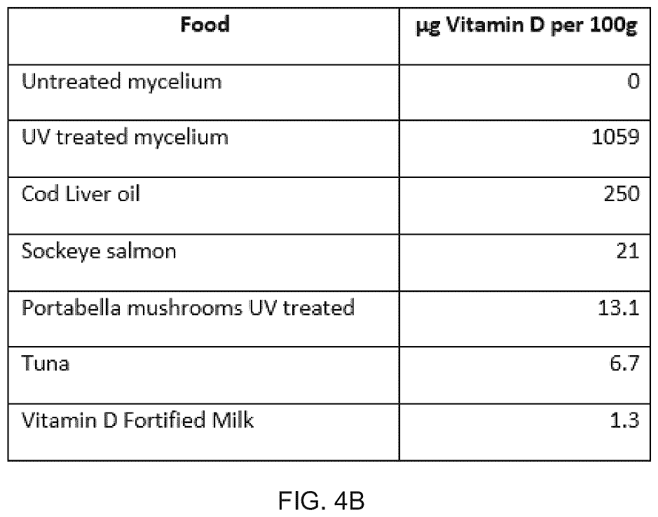

FIG. 4A is a graph of the vitamin D content of various edible products including a UV treated mycelium product and an untreated mycelium product, in accordance with some embodiments. FIG. 4B is a table of the vitamin D content of various edible products, corresponding to FIG. 4A formed using the method 100. Particularly, the UV treated mycelium product was formed from a mycelium product harvested within approximately 45 minutes from the reactor. The harvested mycelium product that was wet, was washed, drained, and then weighed out into about 25 grams portions. The harvested mycelium product was then distributed in an even layer with a thickness in a range of about 0.5 to 1 centimeters (e.g., 0.5 centimeters, 0.6 centimeters, 0.7 centimeters, 0.8 centimeters, 0.9 centimeters, or 1 centimeter, inclusive). A first side of the harvested mycelium product was placed under a UV light for about 5 minutes so as to form an enhanced mycelium product. The distance from the harvested mycelium product to the UV light lamp is approximately 4 centimeters.

Then, the enhanced mycelium product was stirred or flipped to a second side. The second side was exposed to the UV light for about another 5 minutes. The process was repeated with other samples of harvested mycelium products. All of the samples were pooled (e.g., combined, accumulated, etc.) and excess moisture (e.g., water) squeezed out in a mesh bag or any other suitable container. The enhanced mycelium product was be dehydrated to complete dryness at 135° F. (e.g., about 57° C.). After UV light exposure, the enhanced mycelium product did not turn orange or conidiate any more than a control sample. Conidiation is a biological process in which filamentous fungi reproduce asexually from spores. The control sample was a mycelium product that was not exposed to UV light but otherwise subjected to the same processing procedures.

FIGS. 4A-4B illustrate the vitamin D content in micrograms (μg) of vitamin D per 100 g of product of the enhanced mycelium product (e.g., the UV treated mycelium) in comparison to vitamin D fortified milk, tuna, UV treated portabella mushrooms, sockeye salmon, cod liver oil, and untreated mycelium products. As shown in FIG. 4A-4B, the untreated mycelium has about 0 μg of vitamin D, while UV treated mycelium has greater than 1,000 μg per 100 g of UV treated mycelium, demonstrating a substantial increase in vitamin D content after 10 minutes of exposure to UV light. The UV treated mycelium may also have a larger amount of vitamin D content than cod liver oil (250 μg per 100 g), sockeye salmon (21 μg per 100 g), UV treated portabella mushrooms (13.1 μg per 100 g), tuna (6.7 μg per 100 g), and vitamin D fortified milk (1.3 μg per 100 g). In particular, the UV treated mycelium that was subjected to 10 minutes of UV exposure has a vitamin D content of about 80 times that of UV treated portabella mushroom. FIGS. 4A-4B illustrate that the method 100 increases the vitamin D content of mycelium products and that the resulting vitamin D content is substantial (e.g., 4 times more, 50 times more, 100 times more, etc.) in comparison to other vitamin D-containing products.

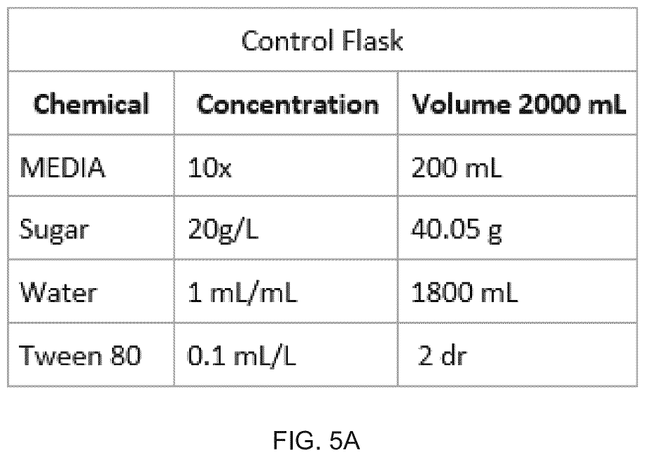

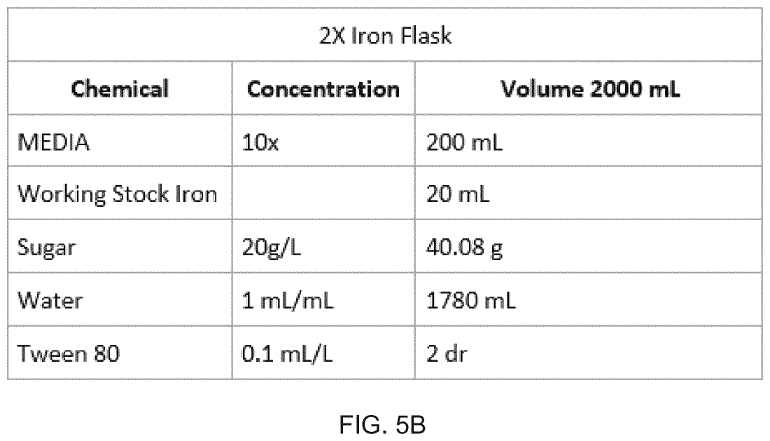

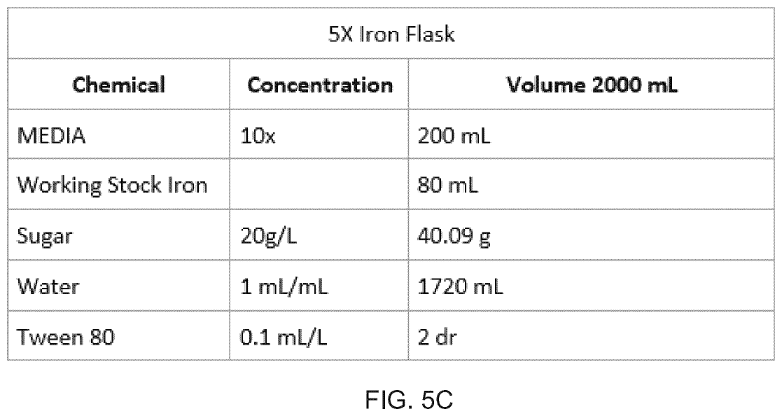

FIGS. 5A-5C are tables of the composition of a control growth media, a growth media with 2X iron content, and a growth media with 5X iron content, respectively, for fungal mycelium, in accordance with some embodiments. In FIG. 5A, fungal mycelium cultures may be grown in 1× Vogel's Medium as typically done in preparing mycelium masses. Vogel's Medium may be a convenient medium for Neurospora and other ascomycetes. 1× Vogel's Medium may include Na₃ citrate, KH₂PO₄, NH₄NO₃, MgSO₄ 7H₂O, CaCl₂ 2H₂O, citric acid, ZnSO₄ 7H₂O, Fe(NH₄)₂ 6H₂O, CuSO₄ 5H₂O, MnSO₄ 4H₂O, H₃BO₃, Na₂MoO₄ 2H₂O, distilled water, glucose, thiamine hydrochloride solution, biotin solution, any combination thereof, or any other suitable materials. For example, Vogel's Medium may include 2% Vogel's 50× salts, 0.01% trace elements solution, 0.005% biotin, and 1.0% glucose for batch submerged culture experiments.

As shown in FIG. 5A, the control media with a volume of about 2,000 mL may include about 200 mL of Vogel's Medium at a concentration of 10× then diluted to a concentration of 1× for use, about 40.05 g of sugar at a concentration of 20 g/L, about 1,800 mL of water at a concentration of about 1 mL/mL, and about 2 drops of TWEEN 80® at a concentration of about 0.1 mL/L. TWEEN 80® is a polysorbate surfactant which helps in stabilizing protein and in protein membrane studies. TWEEN 80® can be used for the extraction of membrane proteins and isolation of nuclei form cells. Fungal mycelium cultures of N. crassa (Neurospora crassa) may be grown in the control growth media for about 24 hours, harvested in a cheesecloth bag, washed, and then dehydrated overnight.

FIGS. 5B-5C illustrate tables of the 2× iron content growth media and the 5× iron content growth media, respectively, for fungal mycelium cultures, in accordance with some embodiments. For both the 2× iron content growth media and the 5× iron content growth media a stock solution of ammonium iron (II) sulfate hexahydrate in distilled water may be prepared with a concentration of about 1 g/100 mL of actual solution. This may be equivalent to trace metals stock concentration which is a common stock solution used for all the growth media. The stock solution may then be diluted to about 5 mL of solution in about 500 mL distilled water to make a working stock solution which is equivalent to 100× of Vogel's Medium's amount of iron. In FIG. 5B, about 20 mL of the working stock solution may be used to prepare the 2× iron content growth media. In FIG. 5C, about 80 mL of the working stock solution may be used to prepare the 5× iron content growth media.

In FIG. 5B, the 2× iron amount growth media with a volume of about 2,000 mL may include 200 mL of Vogel's Medium at a concentration of 10× then diluted to a concentration of 1× for use, about 20 mL of working stock iron, about 40.08 g of sugar at a concentration of about 20 g/L, about 1780 mL of water at a concentration of about 1 mL/mL, and about 2 drops of TWEEN 80® at a concentration of about 0.1 ml/L. In FIG. 5, the 5× iron amount growth media with a volume of about 2,000 mL may include 200 mL of Vogel's Medium at a concentration of 10× then diluted to a concentration of 1× for use, about 80 mL of working stock iron, about 40.09 g of sugar at a concentration of about 20 g/L, about 1720 mL of water at a concentration of about 1 mL/mL, and about 2 drops of TWEEN 80® at a concentration of about 0.1 mL/L.

The 2× iron amount growth media of FIG. 5B and the 5× iron amount growth media of FIG. 5C may then be inoculated with conidia and growth for 48 hours in a shaking incubator at about 0° C. After 48 hours, mycelium masses may be harvested by filtering through a mesh bag or any other suitable porous container. The mycelium masses may then be rinsed with water for one minute and then squeezed dry. The collected mycelium masses may then be dehydrated at about 52° C. (125° F.) for about 24 hours to reach complete dryness. The mycelium masses may then be ground to a fine powder for analysis. In some embodiments, mycelium masses may be formed by the method 200 from the control growth media, the 2× iron amount growth media, and the 5× iron amount growth media. The 5× iron amount growth media may result in a higher iron content at 12.2 mg of iron per 100 g of mycelium mass than the 2× iron amount growth media at 10.01 mg of iron per 100 g of mycelium mass and the control growth media at 4.32 mg of iron per 100 g of mycelium mass. The 2× iron amount growth media may result in a higher iron content than the control growth media.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings and tables in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations.

Thus, particular implementations of the invention have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

As used herein, the singular forms “a”, “an”, and “the” include plural referents unless the context clearly dictates otherwise. Thus, for example, the term “a member” is intended to mean a single member or a combination of members, “a material” is intended to mean one or more materials, or a combination thereof.

As used herein, the terms “about” and “approximately” generally mean plus or minus 10% of the stated value. For example, about 0.5 would include 0.45 and 0.55, about 10 would include 9 to 11, about 1000 would include 900 to 1100.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Other substitutions, modifications, changes and omissions may also be made in the design, operating conditions and arrangement of the various exemplary embodiments without departing from the scope of the present invention.

Reference throughout this specification to “one embodiment,” “an embodiment,” or similar language means that a particular feature, structure, or characteristic described in connection with the embodiment is included in at least one embodiment of the present disclosure. Appearances of the phrases “in one embodiment,” “in an embodiment,” and similar language throughout this specification may, but do not necessarily, all refer to the same embodiment. Similarly, the use of the term “implementation” means an implementation having a particular feature, structure, or characteristic described in connection with one or more embodiments of the present disclosure, however, absent an express correlation to indicate otherwise, an implementation may be associated with one or more embodiments.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings and tables in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated in a single software product or packaged into multiple software products.

Thus, particular implementations of the invention have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A method of preparing an edible meat substitute product, comprising:
providing a mycelium product comprising fungal mycelium; and
exposing the mycelium product to a light having a wavelength in a range of 100 nanometers to 400 nanometers for a period of time so as to provide an enhanced mycelium product having a vitamin D content of greater than 1 μg per 100 grams of the enhanced mycelium product.

2. The method of claim 1, wherein the period of time is in a range of 1 second to 300 seconds.

3. The method of claim 2, wherein the period of time is in a range of 25 seconds to 45 seconds.

4. The method of claim 1, wherein the mycelium product has a moisture content of greater than 90 wt %.

5. The method of claim 1, wherein the mycelium product is compacted so as to have a moisture content of less than 80 wt %.

6. The method of claim 1, wherein the mycelium product is dehydrated so as to have a moisture content of less than 10 wt %.

7. The method of claim 1, wherein exposing the mycelium product to the light comprises:
flowing the mycelium product through a transparent conduit; and
exposing the mycelium product to the light through the transparent conduit.

8. The method of claim 1, wherein exposing the mycelium product to the light comprises exposing a first side of the mycelium product to the light, the method further comprising:
exposing a second side of the mycelium product to the light for a second period of time.

9. The method of claim 8, wherein the second period of time is in a range of 1 second to 300 seconds.

10. The method of claim 1, wherein the mycelium product is at a distance in a range of 1 centimeter to 10 centimeters from a source of the light.

11. The method of claim 1, wherein the wavelength is in a range of 280 nanometers to 315 nanometers.

12. The method of claim 1, wherein the fungal mycelium comprises fungi from the genus Neurospora.

13. The method of claim 12, wherein the fungal mycelium comprises fungi from the species Neurospora crassa.

14. The method of claim 1, wherein the enhanced mycelium product has a vitamin D content of from about 1 microgram per 100 grams of the enhanced mycelium product to about 1 milligram per 100 grams of the enhanced mycelium product.

15. A method of preparing an edible meat substitute product, comprising:
providing a growth media having an iron amount in a range of from 0.1 mg/L to 10 mg/L;
growing fungal cells in the growth media to produce a mycelium mass, the mycelium mass having an iron content greater than or equal to 5 mg of iron per 100 g of mycelium mass; and
separating the mycelium mass from the growth media.

16. The method of claim 15, wherein the iron amount is in a range of 0.1 mg/L to 2 mg/L.

17. The method of claim 15, further comprising:
adding at least one of a sugar, a nitrogen-containing compound, or a phosphate-containing compound to the growth media.

18. The method of claim 15, further comprising:
adding a second growth media to the mycelium mass, the second growth media having a second iron amount in a range of 0.1 mg/L to 10 mg/L.

19. The method of claim 18, further comprising:
adding at least one of a sugar, a nitrogen-containing compound, or a phosphate-containing compound to the second growth media.

20. The method of claim 15, wherein the mycelium mass comprises fungi from the genus Neurospora.

21. The method of claim 20, wherein the mycelium mass comprises fungi from the species Neurospora crassa.

22. The method of claim 15, wherein the mycelium mass has an iron content of from 5 mg of iron per 100 g of mycelium mass to 15 mg of iron per 100 g of mycelium mass.

23. A method of preparing an edible meat substitute product, comprising:
providing a growth media having an iron amount in a range of 0.1 mg/L to 10 mg/L;
growing fungal cells in the growth media to produce a mycelium mass, the mycelium mass having an iron content greater than or equal to 5 mg of iron per 100 g of mycelium mass
separating the mycelium mass from the growth media; and
exposing the mycelium mass to a light having a wavelength in a range of 100 nanometers to 400 nanometers for a period of time so as to form an enhanced mycelium product having a vitamin D content of greater than 1 μg per 100 grams of the enhanced mycelium product.

24. The method of claim 23, wherein the period of time is in a range of 1 second to 300 seconds.

25. The method of claim 23, wherein the iron amount is in a range of 0.1 mg/L to 2 mg/L.

26. The method of claim 23, further comprising:
adding at least one of a sugar, a nitrogen-containing compound, or a phosphate-containing compounds to the growth media.

27. The method of claim 23, wherein the mycelium mass comprises fungi from the genus Neurospora.

28. The method of claim 27, wherein the mycelium mass comprises fungi from the species Neurospora crassa.

29. The method of claim 23, wherein the enhanced mycelium product has a vitamin D content of from about 1 microgram per 100 grams of the enhanced mycelium product to about 1 milligram per 100 grams of the enhanced mycelium product.

30. The method of claim 23, wherein the mycelium mass has an iron content of from 5 mg of iron per 100 g of mycelium mass to 15 mg of iron per 100 g of mycelium mass.
